(19) United States
(12) Patent Application Publication
(10) Pub. No.: US 2013/0295627 A1
(43) Pub. Date: Nov. 7, 2013

(54) PROCESS FOR PRODUCING METHYL BUTENOL (2-METHYL-3-BUTEN-2-OL)

(71) Applicants: Robert J. Hanchar, Charlotte, MI (US); Susan Kleff, Okemos, MI (US)

(72) Inventors: Robert J. Hanchar, Charlotte, MI (US); Susan Kleff, Okemos, MI (US)

(21) Appl. No.: 13/859,356

(22) Filed: Apr. 9, 2013

Related U.S. Application Data
(60) Provisional application No. 61/621,908, filed on Apr. 9, 2012.

(51) Int. Cl. C12P 7/04

(52) U.S. Cl. CPC C12P 7/04
USPC 435/157

(57) ABSTRACT

Disclosed is a process for producing 2-methyl-3-buten-2-ol (MBO). The process is carried out by fermenting a hydrocarbon in a fermentation medium in the presence of a MBO-producing microorganism to produce the MBO. The MBO is removed from the fermentation medium to maintain a concentration of MBO non-toxic to the MBO-producing microorganism by flowing a gas through or across the fermentation medium to effectively remove at least a portion of the MBO into a vapor phase region of the fermentation medium. The rate of removal is measured according to the amount of MBO maintained in the vapor phase region, which is measured according to the vapor pressure of the MBO in the vapor phase region.

### CROSS REFERENCE TO RELATED APPLICATIONS

This is a non-provisional application based upon U.S. Provisional Application Ser. No. 61/621,908, filed Apr. 9, 2012, which is incorporated herein by reference.

### FIELD OF THE INVENTION

This invention is directed to a process for producing 2-methyl-3-buten-2-ol (MBO). In particular, this invention is directed to a process for producing MBO by fermentation, and using a gas as a driving force to remove the MBO into the vapor phase, while the MBO is maintained at a non-toxic level in the fermentation medium.

### BACKGROUND OF THE INVENTION

2-methyl-3-buten-2-ol (MBO) is a branched, five carbon alcohol that is produced in significant quantities (but at low titers) by pine and other trees. It is typically produced by a chemical process from isoprene for use in a variety of applications (such as fragrances and food flavoring).

MBO can also be produced via fermentation. A fermentation production process requires not only an effective production host and fermentation process, but also the effective recovery and separation of the product from fermentation broth or medium. An effective removal of the fermentation product is especially needed, when the product itself is toxic to the producing organism, as is the case for MBO. In these situations, a continuous recovery method that prevents the buildup of toxic levels of the product would be advantageous, allowing for sustained productivity by the organism.

Removal of MBO from a fermentation broth, however, is hindered by the high water solubility of the MBO. MBO resembles butanol in structure and water solubility. Extensive efforts have been put forth to develop acceptable methods to recover butanol from fermentation broth, all have proven to be in-adequate so far. The recovery of MBO would be expected to face the same challenges.

A fermentation process that produces MBO without reaching toxic levels for the microorganism through the continuous removal of MBO would be advantageous.

### SUMMARY OF THE INVENTION

This invention provides a process for producing MBO. The process enables the MBO to be produced by fermentation, without reaching toxic levels for the microorganism. In particular, the MBO can be removed from the fermentation medium using an inert gas technique. Because MBO is known to be highly soluble in an aqueous medium, removal of the MBO using the inert gas technique would not have been expected to provide such an advantageous result.

According to one aspect of the invention, the process for producing MBO includes a step of fermenting a hydrocarbon in the presence of a MBO-producing microorganism in a fermentation medium to produce the MBO. During fermentation, a gas such as an inert gas is flowed through or across the fermentation medium to effectively remove at least a portion of the MBO into a vapor phase region of the fermentation medium. In one embodiment of the invention, the MBO is removed at a rate such that the MBO is present or is maintained in the vapor phase region at a MBO partial pressure of from 0.1 to 140 mmHg, alternatively from 0.16 to 132 mmHg. Maintaining the MBO in the desired partial pressure range, while flowing the gas through or across the fermentation medium, provides a highly effective method of removing the aqueous-soluble MBO.

In one embodiment of the invention, a vapor stream is removed from the vapor phase region, and the MBO is recovered from the vapor stream. The MBO can be recovered from the vapor stream using any appropriate means. For example, the MBO can be recovered from the vapor stream by condensation, adsorption or absorption.

According to another aspect of the invention, the MBO-producing microorganism is an organism that actively expresses an MBO synthase. The fermentation of the hydrocarbon can be carried out according to any appropriate pathway. For example, the MBO can be produced from dimethylallyl pyrophosphate (DMAPP), which can be produced by at least one pathway selected from the group consisting of the MVA pathway or the MEP pathway.

### BRIEF DESCRIPTION OF THE DRAWINGS

Examples of various preferred embodiments of this invention are shown in the attached Figures, wherein:

FIG. 1 is a chart illustrating the rate of removal of MBO from a fermentation medium by using a gas as a driving force for the removal; and

FIG. 2 is a chart illustrating the rate of removal of MBO according to this invention, compared to the predicted rate according to Henry's constant.

### DETAILED DESCRIPTION OF THE INVENTION

### Introduction

This invention is directed to the production of MBO, a five carbon alcohol with high solubility in aqueous culture medium. The process enables the continuous removal of the MBO from the fermentation broth or medium via gas stripping into the exhaust gas and recovering the MBO from the exhaust gas. The process is beneficial in that it allows continuous separation, recovery, and purification of the MBO fermentation product.

The process is carried out by fermenting a hydrocarbon in a fermentation medium in the presence of a MBO-producing microorganism to produce the MBO. The MBO is removed from the fermentation medium to maintain a concentration of MBO non-toxic to the MBO-producing microorganism by flowing a gas through or across the fermentation medium to effectively remove at least a portion of the MBO into a vapor phase region of the fermentation medium. The rate of removal of MBO from the fermentation medium is measured according to the amount of MBO maintained in the vapor phase region, which is measured according to the vapor pressure of the MBO in the vapor phase region.

### Production of MBO

MBO can be produced by a variety of organisms as metabolites in the carotenoid and isoprenoid pathways. Examples of natural producers are plants, mainly pine and oak trees. MBO can also be produced either via the mevalonate (MVA) or the non-mevalonate pathway, also known the 2-C-methyl-D-erythritol 4-phosphate/1-deoxy-D-xylulose 5-phosphate pathway (MEP/DOXP pathway) of isoprenoid biosynthesis. Both lead to the formation of the precursors isopentenyl pyrophosphate (IPP) and dimethylallyl pyrophosphate (DMAPP).

The MVA pathway, or HMG-CoA reductase pathway, is present in all higher eukaryotes and many bacteria and is needed in the synthesis of cell membranes and hormones.

The MEP pathway is the main producer of terpenoids in plants. A significant number of algae and bacteria synthesize IPP and DMAPP via the non-mevalonate pathway.

In particular, MBO can be produced from DMAPP through the action of the enzyme MBO synthase. According to one aspect of the invention, MBO is produced by fermenting a carbohydrate feedstock in the presence of a microorganism that actively expresses MBO synthase enzyme. Fermentation is preferably carried out under conditions in which the microorganism produces the precursor DMAPP by way of at least one pathway selected from the group consisting of the MVA pathway and the MEP pathway. A genetically modified host organism that produces high levels of DMAPP combined with the expression of a suitable MBO synthase enzyme will convert a significant portion of the DMAPP to MBO.

The MBO-producing organism is preferably cultured in a fermentation tank containing a medium comprising a suitable Carbon (C) source and Nitrogen (N) source, as well as other nutrients required for the growth of the organism and the production of MBO. C and N (and other nutrients) can be added to fermentation in a simple batch mode, fed batch mode or continuous mode. The fermentation is carried out at a temperature suitable for the growth of the organism, between 25° C. and 70° C. Gas, such as air, or any one or more gases containing oxygen, nitrogen, and/or carbon dioxide is sparged (i.e., flowed through or across the fermentation medium) at an effective sparge rate, which is a sparge rate for effectively removing MBO from the aqueous fermentation medium, such as for example 0.01 v.v.m to 3.0 v.v.m. The pH is maintained in a suitable range by base/acid addition suitable to maintain growth of the organism and production of MBO.

Fermentation can be aerobic or anaerobic. In a particular embodiment, fermentation is aerobic, and the gas used to remove the MBO from the fermentation medium is an oxygen-containing gas; for example, air.

Examples of microorganisms that are capable of producing MBO via fermentation include gram-positive bacterial cells, Streptomyces cells, gram-negative bacterial cells, Pantoea cells, fungal cells, filamentous fungal cells, Trichoderma cells, Aspergillus cells, or yeast cells; more specifically Escherichia cells (E. coli), Panteoa sp. (P. citrea), Bacillus sp. (B. subtilis), Yarrowia sp. (Y. lipolytica), and Trichoderma (T. reesei), and Fusarium, and Gibberella sp. Additional examples include Saccharomyces cerevisiae, Klebsiella oxytoca, Synechococcus sp., Synechocystis sp., Anabaena sp., Chlorella sp. Scenedesmus sp., Bracteococcus sp. Chlamydomonus sp., C5- or C6-fermentative organisms (including Zymomonas (e.g., Zymomonas mobilis)) or combinations thereof.

Examples of substrates for culturing a microorganism for MBO production (i.e., fermentation of substrate to produce MBO) include hydrocarbons selected from the group consisting of glucose, glycerol, glycerine, dihydroxyacetone, yeast extract, biomass, molasses, sucrose, and oil.

### Removal and Recovery of MBO

To remove the MBO from the fermentation medium, and maintain a non-toxic MBO concentration in the fermentation medium, a gas is flowed through or across the fermentation medium to effectively remove at least a portion of the MBO into a vapor phase region of the fermentation medium. For example, fermentation can be carried out in a fermentation vessel, with the fermentation vessel having a liquid phase region and a vapor phase region.

The gas used to remove the MBO from the fermentation medium in the liquid phase region can be an inert gas, meaning a gas that does not substantially reduce or negatively affect the production of MBO by the host organism in the fermentation medium, nor chemically reacts with the MBO. In a preferred embodiment, the inert gas is one that effectively enhances MBO production. For example, an oxygen-containing gas can be used to remove MBO as well as enhance MBO production in an aerobic fermentation process, while a carbon dioxide-containing gas can enhance MBO production in an anaerobic fermentation process, with neither gas chemically reacting with the MBO. Examples of inert gas include, but are not limited to an oxygen-containing gas, a nitrogen-containing gas and a carbon dioxide-containing gas. Air is an example of gas containing both oxygen and nitrogen, as well as a minor quantity of carbon dioxide.

In one embodiment of the invention, the MBO is removed at a rate such that the MBO is present in the vapor phase region at a MBO partial pressure of from 0.1 to 140 mmHg, alternatively from 0.16 to 132 mmHg.

In another embodiment of the invention, the gas is flowed through or across the fermentation medium at a rate of not greater than 3.5 vvm (the rate of gas flow in volume per minute necessary to remove 1 g/L-h at different MBO concentrations), preferably at least 0.01 vvm.

In yet another embodiment, the gas is flowed through the fermentation medium at a rate of not greater than 3 vvm, alternatively not greater than 2 vvm or 1 vvm; and preferably at least 0.01 vvm.

Fermentation can be carried out in any vessel suitable for maintaining MBO in the vapor phase region at the desired level. The MBO can be removed from the vapor phase region for collection and recovery. For example, a vapor stream can be removed from the vapor space, while maintaining the level of MBO in the vapor phase region at the desired vapor pressure range. The MBO in the removed vapor stream can then be recovered by any appropriate means, such as by condensation, absorption or adsorption.

### Uses of MBO

The recovered MBO can be relatively easily converted to isoprene through any appropriate means. The isoprene can be converted, if desired, into any number of compounds having a wide variety of uses. The MBO can also be used directly as a fuel. Biological pathways that can be engineered into microorganisms are known and may allow the fermentative production of MBO.

### EXAMPLES

### Example 1

### Stripping of MBO from an Aqueous System Using Gas-Sparging and Recovery Via a Cold Trap

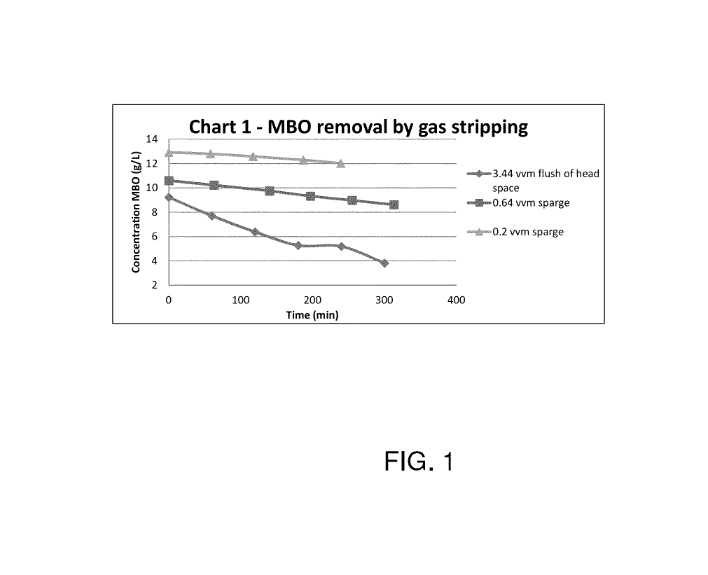

A 3.3 L total volume fermentor vessel was filled with 2.5 L of an aqueous solution containing 10 g/L 2-methyl-3-buten-2-ol (MBO). The solution was incubated at 37° C., agitated at 750 rpm and sparged (at 0.64 vvm) with air through a sparge in the base of the fermentor. Samples were removed periodically from the fermentation vessel and analyzed for residual MBO by HPLC. The removal of MBO was measured by subtraction of residual MBO from the initial concentration. The change in MBO concentration in the aqueous solution using this method is shown in Chart 1 (FIG. 1). The exhaust gas from the fermentor was passed through a cold trap cooled in an ice water bath to condense and recover the removed MBO. 4.98 g of MBO were removed from the aqueous solution after 5 hours of air sparging. The cold trap collected 16.3 mL of an aqueous solution, which contained 1.265 g of MBO.

### Example 2

### Stripping of MBO from an Aqueous System Using a Headspace Sweep Gas And Recovery Via a Cold Trap

A 3.3 L fermentor vessel was filled with 2.5 L of a 10 g/L aqueous solution of 2-methyl-3-buten-2-ol (MBO). The solution was incubated at 37° C., agitated at 750 rpm and the headspace was swept with 3.334 vvm (wrt solution volume) air. Samples were removed periodically from the fermentation vessel and analyzed for residual MBO by HPLC. The change in MBO concentration in the vessel is shown in Chart 1 (FIG. 1). The amount of f MBO removed was calculated by subtraction of residual MBO from the initial concentration. The exhaust gas from the fermentor was passed through a cold trap maintained in an ice water bath to condense and recover the removed MBO. 13.55 g of MBO were removed from the aqueous solution after 5 h the head space of the vessel was flushed with air. 63.2 mL of an aqueous solution were condensed in the trap, which contained 1.735 g of MBO.

### Example 3

### Stripping of MBO from Fermentation Culture Medium Using Gas Sparging and Recovery Via a Cold Trap

A 3.3 L total volume fermentor was filled with 1.0 L of fermentation medium (M9) containing 12 g/L of 2-methyl-3-buten-2-ol (MBO). The medium was incubated at 37° C., agitated at 750 rpm and sparged with air (at 0.2 vvm) through a sparge in the base of the fermentor. Samples were removed periodically from the fermentation vessel and analyzed for residual MBO by HPLC. The removal of MBO was calculated by subtraction of residual MBO from the initial MBO concentration. The change in concentration is shown in Chart 1 (FIG. 1). The exhaust gas from the fermentor was passed through a cold trap maintained in an ice water bath to condense and recover the removed MBO. A total of 0.90 g of MBO was removed from the aqueous solution in the 4 h the sample was sparged with air. 1.72 mL of condensate was collected that contained 0.306 g of MBO.

Chart 1 (FIG. 1) illustrates that it is possible to remove MBO from an aqueous solution, and it is possible to remove more than 1 g/L-h by flushing the headspace with air. The efficiency of MBO removal by flushing the head space would decrease at larger scale, since the ratio of fermentation liquid volume to surface area would presumably increase greatly at a commercial scale. It would be expected that a better mass transfer would be obtained by sparging the aqueous broth, but there will be a practical limit as to how much air can be sparged through a fermentor. A reasonable sparge rate limit through a fermentation is 1 vvm.

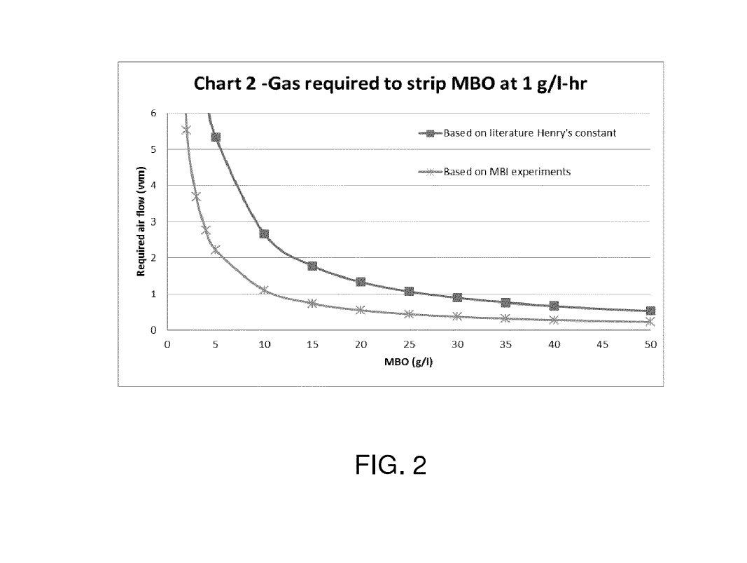

Chart 2 (FIG. 2) compares the sparge rate necessary to remove 1 g/L-h at different MBO concentrations based on the Henry's constant stated in the literature, compared to the results MBI obtained.

To avoid the toxic product buildup the removal rate has to equal or exceed the productivity. Our data showed MBO can be effectively stripped at a rate of 1 g/L-h with only 1 vvm of gas from a 10 g/L MBO solution. Based on the Henry constant in the literature it would be expected that at the same titer of 10 g/L MBO a sparge rate of >2.5 vvm would be required to strip MBO at a rate of 1 g/L-h, an uneconomical and impractical rate.

In the examples given the product was recovered from the exhaust gas stream by condensation. However, substantially equivalent recovery techniques can be carried out, such as but not limited to trapping on carbon or dissolution into an organic solvent via scrubbing the exhaust stream, could also be used to recover the MBO product from the exhaust gas stream.

The principles and modes of operation of the disclosed techniques have been described above with reference to various exemplary and preferred embodiments. As understood by those of skill in the art, the overall techniques, as defined by the claims, encompasses other related embodiments not specifically enumerated herein.

1. A process for producing 2-methyl-3-buten-2-ol (MBO), comprising the steps of:
fermenting a hydrocarbon in a fermentation medium in the presence of a MBO-producing microorganism to produce the MBO; and
flowing a gas through or across the fermentation medium during fermentation to remove at least a portion of the MBO from the fermentation medium and into a vapor phase region, wherein the MBO is removed at a rate to maintain the vapor phase region at a MBO partial pressure of from 0.1 to 140 mmHg.

2. The process of claim 1, wherein the MBO is removed at a rate to maintain the vapor phase region at a MBO partial pressure of from 0.16 to 132 mmHg.

3. The process of claim 1, wherein a vapor stream is removed from the vapor phase region and the MBO is recovered from the vapor stream.

4. The process of claim 3, wherein the MBO is recovered from the vapor stream by adsorption, absorption, or condensation.

5. The process of claim 1, wherein the microorganism actively expresses a MBO synthase.

6. The process of claim 1, wherein fermentation of the hydrocarbon is carried out by at least one pathway selected from the group consisting of the MVA pathway or the MEP pathway.
